(19) United States
(12) Patent Application Publication
(10) Pub. No.: US 2025/0360116 A1
(43) Pub. Date: Nov. 27, 2025

(54) PICOLINIC-2-CARBOXAMIDE HYBRIDIZED WITH ANTHRAQUINONE DERIVATIVES, METHODS OF PREPARATION, AND A PHARMACEUTICAL COMPOSITION COMPRISING THE SAME

(71) Applicant: AL-ZAYTOONAH UNIVERSITY OF JORDAN, Amman (JO)

(72) Inventors: Tareq Musbah Moh'd AL QIRIM, Amman (JO); Yusuf Mohammad Ali AL HIARI, Amman (JO); Hanan Faris Abdelrahman ALASHQAR, Amman (JO); Lama Abdelqader Moh'd HAMADNEH, Amman (JO)

(73) Assignee: AL-ZAYTOONAH UNIVERSITY OF JORDAN, Amman (JO)

(21) Appl. No.: 19/287,396

(22) Filed: Jul. 31, 2025

Related U.S. Application Data
(63) Continuation-in-part of application No. PCT/JO2023/050009, filed on Oct. 8, 2023.

(30) Foreign Application Priority Data
May 19, 2024 (JO) JOP2024113

(51) Int. Cl. A61K 31/4402; A61K 9/00; A61P 3/06; C07D 213/81

(52) U.S. Cl. CPC A61K 31/4402; A61K 9/0019; A61P 3/06; C07D 213/81

(57) ABSTRACT

A picolinic-2-carboxime compound hybridized with anthraquinone derivative(s), may have formula (I), optionally in salt form:

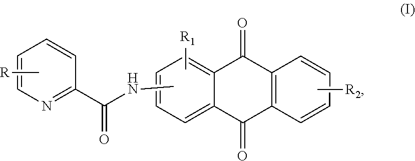

wherein R may be an alkane (e.g., C1-C12), O alkane (e.g., O-C1-C6), OH, NH₂, NHR⁴ with R⁴ being C1-C6, or halogen; R₁ may be an alkane (e.g., C1-C12), O alkane (e.g., O-C1-C6), OH, NH₂, NHR⁴, or halogen; and R₂ may be a halogen (F, Cl, I, Br), alkane (C1-12), OH, NH₂, NHR⁴, CN, COOH, NO₂, COOR⁴, or CONHR⁴. Such a picoline derivative can be made, provided in a pharmaceutical composition, and/or used for treating hyperlipidemia.

### CROSS-REFERENCE TO RELATED APPLICATIONS

The present application is a bypass continuation-in-part of international application PCT/JO2023/050009 filed on Oct. 8, 2023, and claims the benefit of the filing date thereof.

### TECHNICAL FIELD

The present disclosure relates to hybridized compounds, and more particularly to picolinic-2-carboxime compound hybridized with anthraquinone derivatives, pharmaceutical compositions comprising the same, methods of preparation thereof, and methods for treating hyperlipidemia.

### BACKGROUND INFORMATION

Hyperlipidemia is defined by increased of one or more constituent of lipid profile, such as total cholesterol (“TC”), low density lipoprotein cholesterol (“LDL-C”), plasma triglyceride (“TG”) concentration, and decrease in high density lipoprotein cholesterol (“HDL-C”), this condition is considered the major risk factor of atherosclerosis and for coronary artery diseases (“CAD”).

Hyperlipidemia management is based on behavioral changes in diet and changes in therapeutic lifestyles. Changes in the therapeutic lifestyle require a greater focus on physical activity, dietary changes to lower levels of LDL-C, avoidance of smoking, and weight loss, in addition to update of antioxidants. Antioxidants are naturally present in carrots and green vegetables, though they can lateness or interception of plaque formation. When dietary and therapeutic lifestyle changes are ineffective in reducing high lipid levels, there may be a need a pharmacological treatment.

For instance, pharmacological treatment of hyperlipidemia includes administration of statins to patients having hyperlipidemic profile. These statins include Simvastatin, Lovastatin, Pravastatin, Atorvastatin, Fluvastatin, Pitavastatin, and Rosuvastatin; however, these hyperlipidemic agents are not suitable for patients with high levels of liver transaminase and myopathy, as myopathy may progress to rhabdomyolysis and kidney failure.

As another pharmacological treatment of hyperlipidemia, resins such as bile acid sequestrants are anion-exchange resins are used. Such resins act by reducing serum cholesterol levels through binding to the bile acids in the intestine and avoid them reabsorbed into the blood. Although resins inhibit LDL-C levels, they may raise the level of triglycerides.

Alternatively, nicotinic acid, a water-soluble carboxylated pyridine derivative is used as an antihyperlipidemic agent. nicotinic acid reduces the synthesis of TGs in the liver through inhibiting both the synthesis and esterification of fatty acids, thereby enhances apo B degradation. The benefit of nicotinic acid is attributed to its ability to lower the level of TGs synthesis and decreasing very-low-density lipoproteins production, as a result, reduced LDL-C levels. However, nicotinic acid causes flushing, headache, dizziness, blurred vision and gastrointestinal problems.

Additionally, a study conducted by Abu Farha et al. (2017) provided isonicotinic carboxamide derivatives, namely N-(benzoylphenyl)pyridine-4-carboxamide derivatives, and their use as antihyperlipidemic agents.

Moreover, a study conducted by Siddamurthi et al. (2020) provided different anthraquinone analogs and their potential pharmacological effects, and methods of synthesizing anthraquinone.

### SUMMARY

Therefore, it is an object of the present disclosure to provide picolinic-2-carboxime compound hybridized with anthraquinone derivatives, according to the general formula (I), or salts thereof:

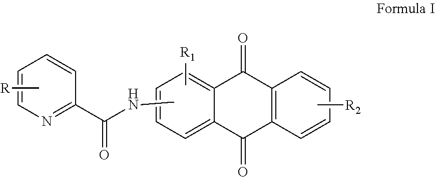

Wherein R may be selected from a group including alkane (e.g., C1-C12), O alkane (e.g., C1-C6), OH, NH₂, NHR⁴, NO₂, or halogen, R⁴ being C1 to C6, preferably methyl, ethyl, propyl, isopropyl, butyl, isobutyl, sec-butyl, or tert-butyl;
R₁ may be selected from a group including alkane (e.g., C1-C12, or C1-C6), O alkane (C1-C6), OH, NH₂, NHR⁴, or halogen; and
R₂ may be selected from a group including halogen (e.g., F, Cl, I, Br), alkane (e.g., C1-12), OH, O(C1-C6), NH₂, NHR⁴, CN, COOH, NO₂, COOR₄, or CONHR⁴.

It is another object of the present disclosure to provide a method of preparing the compound of the general formula (I), wherein such method may include the steps of:
Mixing picolinic acid and thionyl chloride in toluene to produce a first mixture;
Refluxing the first mixture for about 24 hours at a temperature of about 80° C. till completion of reaction;
Adding Toluene to the refluxed first mixture to produce a second mixture, then evaporating the second mixture under vacuum to remove thionyl chloride and produce picolinoyl chloride;
Adding picolinoyl chloride to an anthraquinone derivative, pyridine, and trimethylamine to produce a third mixture;
Heating the third mixture at a temperature of about 100° C. to produce a solid mixture;
Cooling down the solid mixture and adding cold water to the solid mixture while stirring, followed by adjusting the pH to about 10 using potassium carbonate to remove excess picolinoyl chloride, and to provide a fourth mixture; and
Performing suction filtration to the fourth mixture in order to provide a fifth mixture.

In some aspects of the present disclosure, the anthraquinone derivative may include 1-amino-anthraquinone.

In other aspects of the present disclosure, the anthraquinone derivative may include 2-amino-anthraquinone.

In yet other aspects of the present disclosure, the anthraquinone derivative may include 1-amino-4-hydroxy-amino-anthraquinone.

It is another object of the present disclosure to provide a pharmaceutical composition including the general formula (I) and/or pharmaceutically acceptable salts thereof and a pharmaceutically acceptable carrier/excipient.

In some aspects, the pharmaceutical composition may be formulated as a solid, liquid, or semi-solid dosage form.

In some aspects, the pharmaceutical composition may be administered via different routes such as oral, parenteral, intramuscular, intranasal, sublingual, intratracheal, ocular, vaginal, rectal, or intraventricular.

It is yet another object of the present disclosure to provide a method for treating hyperlipidemia comprising administering a therapeutically effective amount of the pharmaceutical composition.

### BRIEF DESCRIPTION OF THE DRAWINGS

The disclosure will now be described with reference to the accompanying drawings, which illustrate embodiments of the present disclosure, without however restricting the scope of the disclosure thereto, and in which:

FIG. 1 illustrates a flowchart of a method of preparing a compound of general formula (I), the method being configured in accordance with embodiments of the present disclosure.

FIG. 2 illustrates a bar chart showing effect of Triton WR-1339 on plasma lipid levels of Wistar rats after about 6 hours of administration, wherein values are expressed as mean±SEM from five rats in each group.

FIG. 3 illustrates a bar chart showing effect of treatment of Wistar Rats using compounds C1, C2, C3 after injection with Triton WR-1339 on plasma TG after about 6 hours of treatment, wherein values are expressed as mean±SEM from five rats in each group.

FIG. 4 illustrates a bar chart showing effect of treatment of Wistar Rats using compounds C1, C2, C3 after injection with Triton WR-1339 on plasma TC after about 6 hours of treatment, wherein values are expressed as mean±SEM from five rats in each group.

FIG. 5 illustrates a bar chart showing effect of treatment of Wistar Rats using compounds C1, C2, C3 after injection with Triton WR-1339 on plasma LDL-C after about 6 hours of treatment, wherein values are expressed as mean±SEM from five rats in each group.

FIG. 6 illustrates a bar chart showing effect of treatment of Wistar Rats using compounds C1, C2, C3 after injection with Triton WR-1339 on plasma HDL-C after about 6 hours of treatment, wherein values are expressed as mean±SEM from five rats in each group.

### DETAILED DESCRIPTION

Embodiments of the present disclosure provide a novel picolinic-2-carboxime compound hybridized with anthraquinone derivatives, according to the general formula (I), or salts thereof:

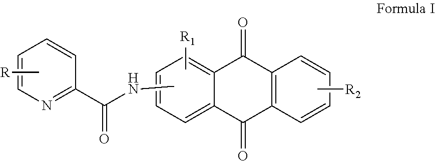

Wherein R may be selected from a group including alkane (e.g., C1-C12), O alkane (e.g., C1-C6), OH, NH₂, NHR⁴, NO₂, or halogen, R⁴ being C1 to C6, preferably methyl, ethyl, propyl, isopropyl, butyl, isobutyl, sec-butyl, or tert-butyl;
R₁ may be selected from a group including alkane (e.g., C1-C12), O alkane (e.g., C1-C6), OH, NH₂, NHR⁴, or halogen; and
R₂ may be selected from a group including halogen (e.g., F, Cl, I, Br), alkane (e.g., C1-12), OH, O (C1-C6), NH₂, NHR⁴, CN, COOH, NO₂, COOR⁴, or CONHR⁴.

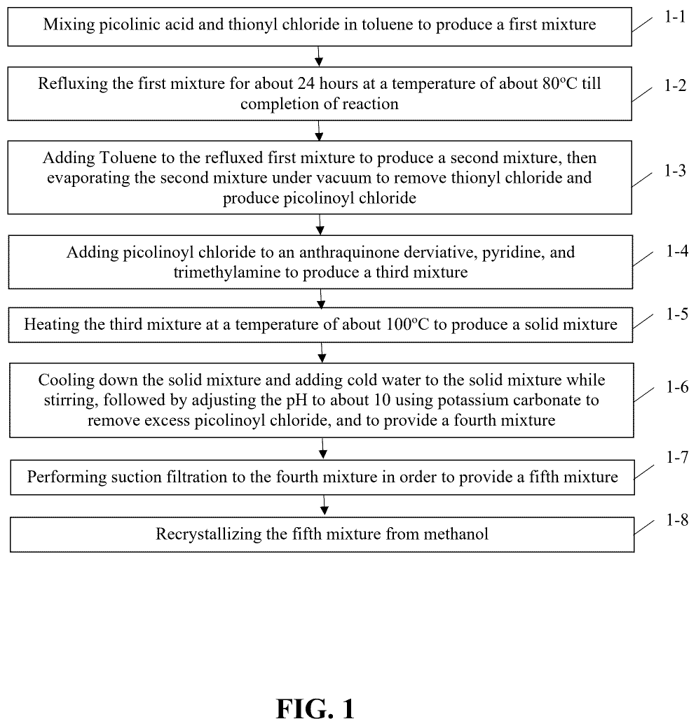

Reference now is being made to FIG. 1 which illustrates a flowchart of a method of preparing the compound of the general formula (I) of the present disclosure according to embodiments of the present disclosure. As illustrated in FIG. 1, the method includes the steps of:
Mixing picolinic acid and thionyl chloride in toluene to produce a first mixture (process block 1-1);
Refluxing the first mixture for about 24 hours at a temperature of about 80° C. till completion of reaction (process block 1-2);
Adding Toluene to the refluxed first mixture to produce a second mixture, then evaporating the second mixture under vacuum to remove thionyl chloride and produce picolinoyl chloride (process block 1-3);
Adding picolinoyl chloride to an anthraquinone derviative, pyridine, and trimethylamine to produce a third mixture (process block 1-4);
Heating the third mixture at a temperature of about 100° C. to produce a solid mixture (process block 1-5);
Cooling down the solid mixture and adding cold water to the solid mixture while stirring, followed by adjusting the pH to about 10 using potassium carbonate to remove excess picolinoyl chloride, and to provide a fourth mixture (process block 1-6); and
Performing suction filtration to the fourth mixture in order to provide a fifth mixture (process block 1-7).

In embodiments of the present disclosure, the anthraquinone derivative may include 1-amino-anthraquinone.

In other embodiments of the present disclosure, the anthraquinone derivative may include 2-amino-anthraquinone.

In yet other embodiments, the anthraquinone derivative may include 1-amino-4-hydroxy-amino-anthraquinone.

In other embodiments of the present disclosure, the method may further include recrystallizing the fifth mixture from methanol to provide the compound of the general formula (I) (process block 1-8).

Other embodiments of the present disclosure further provide a pharmaceutical composition including a compound of general formula (I) and/or pharmaceutically acceptable salt thereof, and a pharmaceutically acceptable carrier/excipient.

The term “pharmaceutical composition”, as used herein, is intended to include a compound of general formula (I) and/or a pharmaceutically acceptable salt thereof.

In embodiments of the present disclosure, the pharmaceutical composition can be, for example, in a liquid form, e.g. a solution, syrup, emulsion and suspension, or in a solid form, e.g. a capsule, caplet, tablet, pill, powder and suppository. Granules, semi-solid forms and gel caps are also considered. In case that the pharmaceutical composition is a liquid or a powder, dosage unit optionally is to be measured, e.g. in the dosage unit of a teaspoon.

The pharmaceutical composition in embodiments of the present disclosure can be formulated for oral administration in solid or liquid form, for parenteral injection or for rectal administration. The pharmaceutical composition can be administered to humans and other mammals orally, sublingually, rectally, parenterally, intracisternally, intraurethrally, intraperitoneally, topically (as powder, ointment or drop), as buccal or as an oral or nasal spray. The term “parenterally”, as used herein, refers to modes of administration which include intravenous, intramuscular, intraperitoneal, subcutaneous, intra-articular injection and infusion.

The term “pharmaceutical acceptable carrier/excipient”, as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch; cellulose and its derivatives such as sodium carboxymethyl cellulose, ethyl cellulose and cellulose acetate; powdered tragacanth; binding agents such as hypromellose; disintegrating agents such as crosscarmellose; water; malt; gelatin; talc; excipients such as cocoa butter and suppository waxes; oils such as peanut oil; cottonseed oil; safflower oil, sesame oil, olive oil, corn oil and soybean oil; glycols, such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgement of the formulator.

All components of the pharmaceutical composition have to be pharmaceutically acceptable. The term “pharmaceutically acceptable” means at least non-toxic.

Embodiments of the present disclosure provide a method for treating hyperlipidemia comprising administering a therapeutically effective amount of the pharmaceutical composition.

The disclosure will be further illustrated on the basis of examples and a detailed description from which further features and advantages may be taken. It is to be noted that the following explanations are presented for the purpose of illustrating and description only; they are not intended to be exhaustive or to limit the disclosure to the precise form disclosed.

### Example 1

Preparation of Novel Picolinic-2-Carboxime Compound Hybridized with Anthraquinone Derivatives

Compounds of the general formula (I) can be prepared by the following scheme:

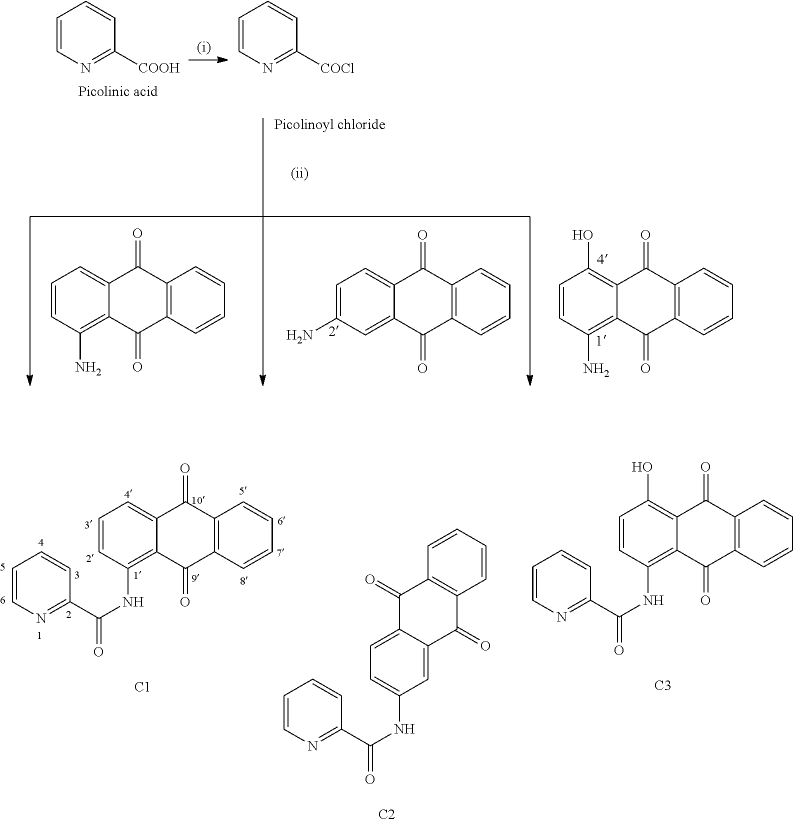

Using a commercially available picolinic acid and thionyl chloride, picolinoyl chloride was prepared as a starting material for the preparation of the compounds (C1-C3) of the general formula (I). The preparation method was initiated by adding a mixture of about 5 g, 40.61 mmol of picolinic acid and about 6 ml, 82.2 mmol of thionyl chloride in about 100 ml of toluene in a flask, the reaction mixture was refluxed for about 24 hours at 80° C. till the completion of the reaction. Then about 20 ml of toluene was added to the mixture twice and evaporated under vacuum to remove excess thionyl chloride and produce about 4 ml and 36.6 mmol of picolinoyl chloride.

For the preparation of compound C1, about 1 ml, 9.16 mmol of picolinoyl chloride was directly added to about 0.71 g, 3.2 mmol of 1-amino-anthraquinone in a 30 ml vial containing about 0.7 ml, 8.65 mmol of pyridine as an acylation catalyst and about 0.7 ml, 5 mmol of trimethylamine as an acid scavenger. The vial was placed in a microwave oven for about 15 minutes at temperature of about 100° C. Upon cooling, about 60 ml of cold water was added to the solid mixture with stirring for about 10 minutes, and the pH was adjusted to 10 using potassium carbonate (K₂CO₃) to remove the excess picolinoyl acid. Suction filtration was carried out to obtain the compound C1. Preparative TLC plates were then used to afford about 0.49 gm, 46.4% of pure compound C1 upon collection and drying the fractions extract, Rf: 0.36 (CHCl₃/MeOH, 95:5); Melting point: 262-265° C.

For the preparation of compound C2, about 1 ml, 9.16 mmol of picolinoyl chloride was directly added to about 0.71 g, 3.2 mmol of 2-amino-anthraquinone in a 30 ml vial containing about 0.7 ml, 8.65 mmol of pyridine as an acylation catalyst and about 0.7 ml, 5 mmol of trimethylamine as an acid scavenger. The vial was placed in a microwave oven for about 15 minutes at temperature of about 100° C. Upon cooling, about 50 ml of cold water was added to the solid mixture with stirring for about 10 minutes, and the pH was adjusted to 10 using potassium carbonate (K₂CO₃) to remove the excess picolinoyl acid. Suction filtration was carried out to obtain the compound C2. Preparative TLC plates were then used to afford about 0.17 g, 15.8% of pure compound C2 upon collection and drying the fractions extract, Rf: 0.40 (CHCl₃/MeOH, 95:5); Melting point: >300° C.

For the preparation of compound C3, about 1 ml, 9.16 mmol of picolinoyl chloride was directly added to about 0.77 g, 3.2 mmol of 1-amino-4-hydroxy-anthraquinone in a 30 ml vial containing about 0.7 ml, 8.65 mmol of pyridine as an acylation catalyst and about 0.7 ml, 5 mmol of trimethylamine as an acid scavenger. The vial was placed in a microwave oven for about 15 minutes at temperature of about 100° C. Upon cooling, about 30 ml of cold water was added to the solid mixture with stirring for about 10 minutes, and the pH was adjusted to 10 using potassium carbonate (K₂CO₃) to remove the excess picolinoyl acid. Suction filtration was carried out to obtain the compound C3. Recrystallization from methanol including charcoal step has furnished about 0.186 g, 16.8% of pure compound C3, Rf: 0.24 (CHCl₃/MeOH, 95:5); Melting point: >300° C.

### Example 2

### Structure Elucidation Data

The 3 synthesized compounds may have chemical characterization as follows:

N-(9,10-dioxo-9,10-dihydroanthracen-1-yl) picolinamide (C1)

¹H-NMR (500 MHz, DMSO-d6): δ=13.93 (brs, 1H, CONH amide), 9.26 (d, J=7.95 Hz, 1H, Ar—H), 8.87 (d, J=4.2 Hz, 1H, Ar—H), 8.28 (d, J=7.0 Hz, 1H, Ar—H), 8.21 (d, J=7.7 Hz, 1H, Ar—H), 8.15 (d, J=6.5 Hz, 1H, Ar—H), 8.09 (d, J=7.27 Hz, 1H, Ar—H), 7.97 (dd, J=4.4, 8.0 Hz, 1H, Ar—H), 7.94 (m, 3H, Ar—H), 7.72 (m, 1H, Ar—H) ppm. IR (KBr disk): ν=3436 (NH amide), 3111, 3065, 1995, 1932, 1790, 1747, 1687 (ketone carbonyl), 1670 (ketone carbonyl), 1630 (amide carbonyl), 1585, 1526, 1478, 1398, 705 cm-1. HRMS (ESI, positive mode): m/z [M+H+] 329.09277 (C20H13N2O3 requires 329.09262).

N-(9,10-dioxo-9,10-dihydroanthracen-2-yl) picolinamide (C2)

¹H-NMR (500 MHz, DMSO-d6): δ=11.21 (brs, 1H, NHCO amide), 8.82 (d, J=1.94 Hz, 1H, Ar—H), 8.73 (d, J=4.4 Hz, 1H, Ar—H), 8.32 (dd, J=2.0, 8.5 Hz, 1H, Ar—H), 8.14 (m, 4H, Ar—H), 8.05 (dd, J=1.3, 7.65 Hz, 1H, Ar—H), 7.85 (2m, 2H, Ar—H), 7.66 (dd, J=5.05, 6.75, 1H, Ar—H) ppm. 13C-NMR (125 MHz, DMSO-d6): δ=182.87 (C═O carbonyl), 181.91 (C═O carbonyl), 163.88 (CONH amide carbonyl), 149.81, 149.02, 144.44, 138.69, 134.99, 134.71, 134.53, 133.59, 128.95, 128.58, 127.82, 127.22, 127.10, 125.68, 123.27, 117.72 ppm. IR (KBr disk): ν=3438 (NHCO amide), 3074, 1725, 1699 (ketone carbonyl), 1679 (ketone carbonyl), 1625 (amide carbonyl), 1592, 1539, 708 cm−1. HRMS (ESI, negative mode): m/z [M−H+] 327.07677 (C20H11N2O3 requires 327.07697).

N-(4-hydroxy-9,10-dioxo-9,10-dihydroanthracen-1-yl) picolinamide (C3)

¹H-NMR (300 MHz, DMSO-d₆): δ=13.89 (br-s, 1H, CONH amide), 13.13 (br-s, 1H, OH), 9.25 (d, J=9.25 Hz, 1H, Ar—H), 8.82 (vr-s, 1H, Ar—H), 8.28, 8.22, 8.18 (3m overlapping, 2H, Ar—H), 8.05 (m, 1H, Ar—H), 7.98 (Br-m, 2H, Ar—H), 7.68 (br-m, 1H, Ar—H), 7.50 (d, J-9.20 Hz, 1H, Ar—H), 5.66 (br-s, 1H, Ar—H) ppm. IR (KBr disk): ν=3405 (CONH amide), 3220 (br OH), 3068, 1958, 1895, 1860, 1757 (ketone carbonyl), 1675 (ketone carbonyl), 1650 (amide carbonyl), 1595, 1533, 1488, 728 cm⁻¹. HRMS (ESI, positive mode): m/z [M+H⁺] 345.08687 (C₂₀H₁₁N₂O₄ requires 345.08753).

### Example 3

### Hyperlipidemic Wistar Rats Preparation

The study was conducted on twenty-five adult male Wistar Rats weighting 200±10 g. Wistar Rats were bred in the animal care center of Faculty of Pharmacy in Al-Zaytoonah University of Jordan. The animals were kept on 12 hours' light/dark cycle, at room temperature (25±1° C.) with free access to food and water. The rats were divided randomly into five groups each consist of five rats, whereby Group I was the normal control group (“NCG”) that received an intraperitoneal administration of normal saline, Group II was the hyperlipidemic control group (“HCG”) that received an intraperitoneal injection of about 200 mg/Kg triton WR-1339 dissolved in about 4% dimethylsulfoxide (“DMSO”)/corn oil, Groups (III, IV, V) rats received intraperitoneal injection of about 200 mg/Kg triton WR-1339.

After about 6 hours of treatment, the rats were anaesthetized with diethyl ether and blood was collected. The blood samples were immediately centrifuged at about 3000 rpm for about 10 minutes and the serum was used for lipid profile measurements (TG, LDL-C, TC and HDL-C) by an automated chemistry analyzer (Model Erba XL-300, Mannheim, Germany). Furthermore, liver samples were collected, and then kept in liquid nitrogen, and stored at (−80° C.) for the mRNA extraction as described in Example 5 below.

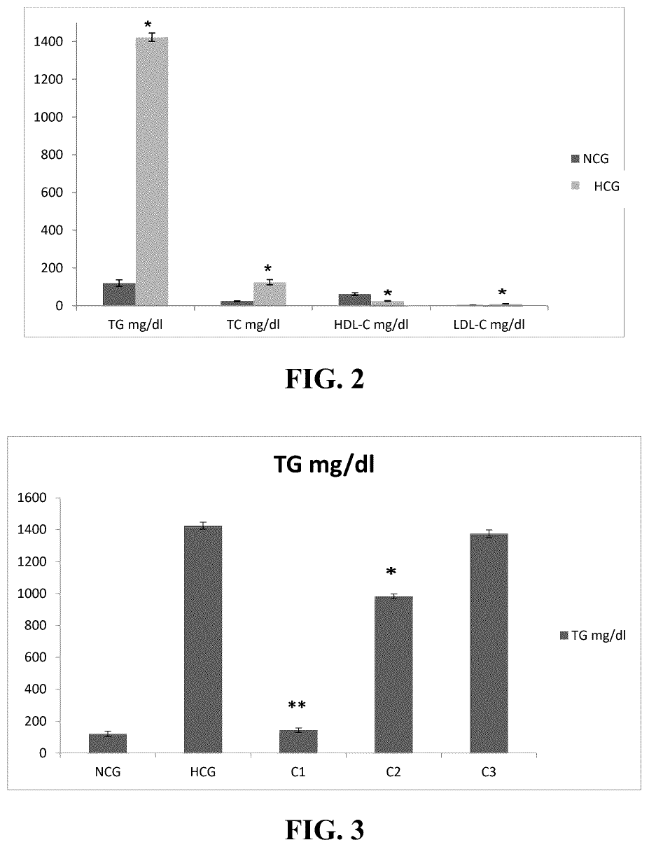

The plasma of TG, TC, LDL-C and HDL-C levels in HCG treated at 6 hours after administration of triton WR-1339 are shown in FIG. 2. As depicted from FIG. 2, Triton WR-1339 caused a significant increase in TG (p<0.05), TC and LDL-C (p<0.05) levels, and a significant decrease in plasma HDL-C level (p<0.05) in HCG at about 6 hours after Triton WR-1339 administration in comparison with the NCG.

### Example 4

### In-vivo Testing

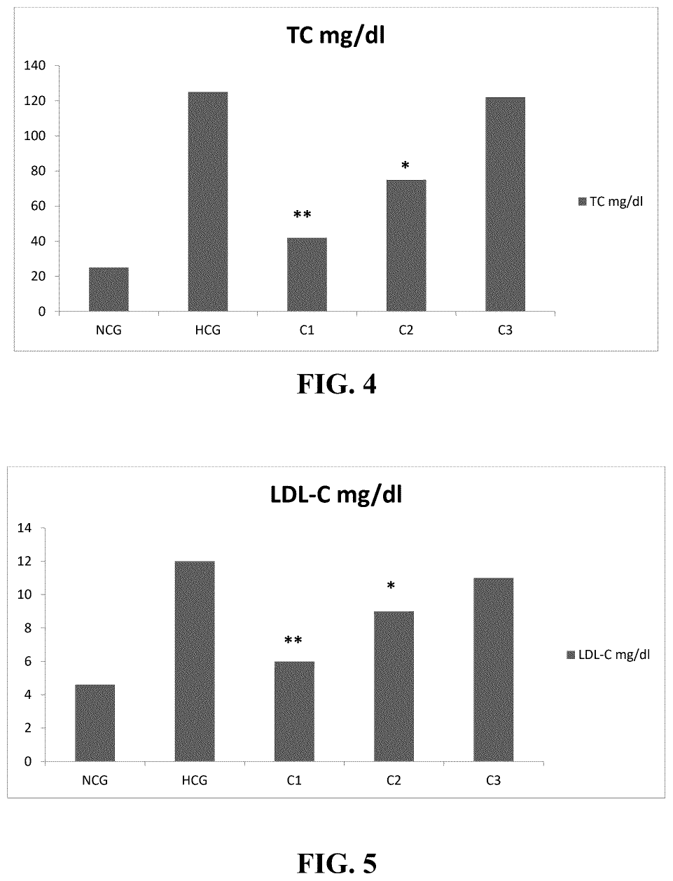

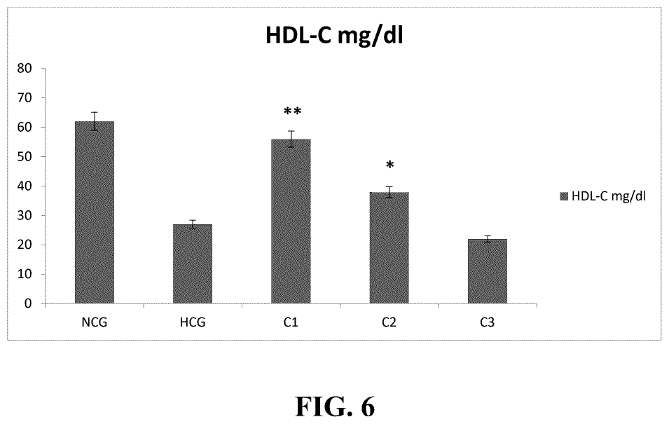

After the injection of Wistar Rats with Triton WR-1339 in Example 3, Groups III, IV, V received an intragastric administration of (20 mg/kg by weight) of compounds C1, C2 and C3, respectively, dissolved in about 4% DMSO/corn oil. FIGS. 3, 4, 5, 6 illustrate bar charts showing the effect of the compounds C1, C2, and C3 on the Triton WR-1339 induced hyperlipidemic Wistar Rats after about 6 hours of treatment on TG, TC, LDL-C, and HDL-C. Similarly, such effect is summarized in Table (1) below.

Table (1): Effect of novel compounds C1, C2, C3 on plasma lipid level in triton WR 1339 induced hyperlipidemia in rats after about 6 hours

| | | | HDL-C | LDL-C |
|---|---|---|---|---|
| Groups | TG mg/dl | TC mg/dl | mg/dl | mg/dl |
| NCG | 120 ± 17.2 | 25 ± 2.2 | 62 ± 7.2 | 4.6 ± 0.5 |
| HCG | 1425 ± 22.6 | 125 ± 14 | 27 ± 1.5 | 12 ± 1 |
| C 1 | 145 ± 14.2 ^{b} | 42 ± 10 ^{b} | 56 ± 4.6 ^{b} | 6 ± 0.7 ^{b} |
| C 2 | 985 ± 15.3 ^{a} | 75 ± 15.2 ^{a} | 38 ± 4.4 ^{a} | 9 ± 0.6 ^{a} |
| C 3 | 1375 ± 23 | 122 ± 11 | 22 ± 2 | 11 ±1.2 |

Values are expressed as mean ± SEM from five animals in each group.
^{a} p < 0.05,
^{b} p < 0.001.

The elevated plasma TG levels induced by the acute injection of triton WR-1339 in Example 3 above were significantly decreased by compound C1 (P<0.001) and compound C2 (P<0.05), while compound C3 showed non-significant effect on TG level in comparison with HCG.

Also, the elevated TC levels were induced by acute injection of triton WR-1339 in Example 3 above were significantly decreased by compound C1 (P<0.001) and compound C2 (P<0.05) while compound C3 showed non-significant effect on TC level in comparison with HCG.

Furthermore, the elevated plasma LDL-C levels induced by acute injection of triton WR-1339 in Example 3 above were significantly lowered by compound C1 (P<0.001) and compound C2 (P<0.05), while compound C3 showed non-significant effect on LDL-C level in comparison with HCG.

Additionally, plasma HDL-C levels were significantly increased by compound C1 (P<0.001) and compound C2 (P<0.05), while compound C3 showed non-significant effect on HDL-C level in comparison with HCG.

### Example 5

### Gene Expression Analysis Using Qrt-PCR

Quantitative assessment of the gene expression profiles from liver tissues of hyperlipidemic samples taken in Example 3 above, has been carried out in duplicates, and using PCR arrays, then CFX 96 real time PCR (Bio-Rad, USA).

Total mRNA Purification

Total mRNA of liver samples was purified using RNeasy® Plus Mini Kit (QIAGEN, U.S.A.). According to the manufacturer's protocol, about 30 mg of liver tissues were lysed using about 600 μg of RLT buffer in order to immediately inactivate RNases, then vortexed for about 30 seconds, and centrifuged at maximum speed for about 3 minutes, followed by the careful removal of supernatant. After that, the samples were transferred to gDNA eliminator spin column, where it was placed in a 2 ml collection tube, allowing efficient removal of genomic DNA, then samples were centrifuged at about 8000×g for about 30 seconds, then discarding the column. After that, about 600 μl of 70% ethanol was added to the flow-through, and mixed well through pipetting up and down, then about 700 μl of the sample was then moved to RNeasy spin column, put in a 2 ml collection tube and that centrifuged at about 8000×g for 15 seconds, then the flow-through has been discarded. Next, about 700 μl of buffer RW1, was added to RNeasy spin column Put in a 2 ml collection tube, and centrifuged at 8000×g for about 15 seconds, then the flow-through has been discarded. After that, about 500 μl of Buffer RPE was added to the RNeasy spin column and centrifuged at about 8000×g for about 15 seconds, and then the flow-through has been discarded. Then about 500 μl Buffer RPE was added to the RNeasy spin column, After that centrifugation for about 2 minutes at about 8000×g. Finally, the RNeay spin column was put in 1.5 ml collection tube, then about 30 μl of RNeasy-free water was added to the RNeasy spin column membrane and centrifuged at about 8000×g for about 1 minute to form an elute of RNA. After that mRNA samples are stored as aliquots of about 10 μl each at about −80° C.

The A260:A280 of the extracted mRNA ranged between about 1.8 to about 2.0, indicating pure RNA samples. Pooled mRNA from samples of each experimental group, ensured that a concentration of about 0.5 μg as a starting mRNA, was used to synthesize cDNA, followed by the applying into RT2 profiler PCR array in duplicates.

cDNA Synthesis using RT² First Strand Kit

For each reverse transcription reaction, a concentration starting from about 0.5 μg mRNA was used. In the first step, the genomic DNA elimination mix was prepared for each pooled mRNA sample that was gently mixed with about 2 μl GE buffer and a complete amount of RNA-free water having about 10 μl total volume. Then, it was incubated in the gDNA elimination mix at for about 5 minutes at about 42° C., and placed immediately in ice for about 1 minute to stop the reaction. The reverse transcription mix has been prepared according to Table (2) below.

TABLE 2
The reverse transcription mix

| Component | Volume |
|---|---|
| 5x buffer BC3 | 4 μl |
| Control P2 | 1 μl |
| RE3 reverse transcription mix | 2 μl |
| RNase-free water | 3 μl |
| Total volume | 10 μl |

After that, about 10 μl of reverse transcription mix was added to each tube that contains about 10 μl of gDNA elimination mix, mixed gently by pipetting up and down, and incubated at about 42° C. for about 15 minutes as a first incubation stage, then immediately incubated at about 95° C. for about 5 minutes as a second incubation stage to stop the reaction. After that, about 91 μl of RNase-free water was added to each reaction, and mixed by pipetting up and down several times. Finally, the reactions were stored at about −20° C. prior to PCR.

Preparation of Samples for Real-time PCR using RT² Profiler PCR Arrays

Real-time PCR was conducted using RT² Profiler PCR arrays (QIAGEN, U.S.A.) in combination with RT² SYBER Green master mixes (QIAGEN, U.S.A.). RT² SYBER Green master mixes have been used to provides accurate real time quantification of RNA targets. The RT² SYBER Green master mix was briefly centrifuged for about 10 seconds to about 15 seconds to move the contents to the bottom of the tube. HotStart DNA Taq Polymerase is activated only after heat activation; therefore, the reactions were prepared at room temperature. After that PCR components mix was prepared, using a 5 ml tube to ensure proper mixing. Table (3) below illustrates the PCR mix components.

TABLE 3
PCR components mix

| Array format | 96-well (A, C, D, F) |
|---|---|
| 2x SYBER Green mastermix | 1350 μl |
| cDNA synthesis reaction | 102 μl |
| RNase-free water | 1248 μl |
| Total volume | 2700 μl |

For each PCR array reaction, about 10 μl of the reverse transcription mix was added to each tube containing about 10 μl of gDNA elimination mix, which was prepared previously, mixed gently by pipetting up and down, and then incubated at about 42° C. for about 15 minutes as a first incubation stage, followed by incubation at about 95° C. for about 5 minutes as a second incubation stage to stop the reaction. After that, about 91 μl of RNase-free water was added to each reaction, and mixed by pipetting up and down several times. Finally, the reaction mixtures were stored in a freezer at about −20° C. till real-time experiments are performed.

### Fatty Acids Metabolism PCR Array

In order to identify the molecular mechanism of the targeted compounds C1, C2, rat fatty acid metabolism profiler (PARN-007ZA) (QUAIGEN, U.S.A.) was used in duplicates.

For each well of 96 wells from each PCR array, about 25 μl of PCR mix was added. After that, CFX96 real time PCR machine (Bio-Rad, U.S.A.) was used according to the following cycling conditions: about 10 minutes at about 95° C., then about 15 seconds at about 95° C., and about 1 minute at about 60° C. for about 40 cycles, with adjusting to the RPM rate at about 1° C./s.

84 genes were analyzed for rat fatty acid metabolism PCR array. Tables (4) and (5) show genes overexpressed and down-regulated in Triton WR-1339 control group. Compounds C1, C2 show overexpressed and down regulation genes in comparison to Triton WR-1339 control group as shown in Table (6) below.

TABLE 4
Genes overexpressed in triton WR-1339
control group in fatty acid metabolism
Genes overexpressed in triton control group
in fatty acid metabolism

| Triton | Fold regulation |
|---|---|
| Cpt1a | 2.29 |
| Decr1 | 3.12 |
| Fabp7 | 3.28 |
| Hadha | 4.78 |
| Lipe | 2.61 |
| Slc27a2 | 2.31 |

TABLE 5
Genes down regulated in triton WR-1339
control group in fatty acid metabolism
Genes overexpressed in triton control group
in fatty acid metabolism

| Triton | Fold regulation |
|---|---|
| Acsl4 | −4.09 |
| Acsm2a | −3.5 |

TABLE 6
Gene expression in Triton, compounds C1 and C2 treated rats
Fold Regulation

| | | Compound | |
|---|---|---|---|
| Fatty Acid Metabolism | Triton 1339 WR | C1 | Compound C2 |
| Acaa2 | 1.74 | −2.78 | 1.04 |
| Acadl | 1.5 | −2.37 | −1.77 |
| Acadvl | 1.2 | −3.34 | −2.08 |
| Acat2 | −1.37 | 2.98 | 2.01 |
| Acot12 | 1.75 | −2.31 | −2.97 |
| Acsl4 | −4.09 | 8.74 | 7.78 |
| Acsm2a | −3.5 | 3.69 | 2.87 |
| Bdh2 | −1.4 | 4.48 | 2.8 |
| Cpt1a | 2.29 | −2.61 | −4.39 |
| Decr1 | 3.12 | −3.88 | −1.97 |
| Fabp4 | −1.38 | 1.73 | 3.55 |
| Fabp7 | 3.28 | −4.43 | −6.64 |
| Hadha | 4.78 | −2.96 | −2.67 |
| Hmgcs1 | 1.44 | 2.14 | 3.86 |
| Lipe | 2.61 | 3.01 | 3.85 |
| Lpl | 1.25 | 4.61 | 5.89 |
| Mcee | 1.25 | −2.54 | −2.13 |
| Slc27a2 | 2.31 | −3.91 | −2.92 |
| Slc27a5 | 1.52 | −1.77 | −2.2 |

As depicted from Table (4), triton control group overexpressed several genes involved in B-oxidation, fatty acid uptake, and cholesterol biosynthesis pathway.

In gene expression of fatty acid metabolism that is involved in peroxisomal β-oxidation pathway, compounds C1 and C2 showed many overexpressed and down regulated genes. For instance, Acetyl-CoA acyltransferase 2 (“Acaa 2”) was found to be down regulated by compound C1, whereas C2 showed no change in fold regulation. The protein produced is responsible for cholesterol ester formation in tissues, and its inhibition is consistently considered as atheroprotective and its inhibition could have a potential protective effect in coronary heart diseases, as reported in the art.

Acyl coenzyme A dehydrogenases, long chain (“Acad1”) was found to be down regulated by compound C1, whereas compound C2 showed no change in fold regulation, the gene encodes an enzyme that catalyzes the initial rate-limiting step in the β-oxidation of fatty acyl-CoA, and its inhibition could have a cardiovascular protective effects, as reported in the art.

Also, acyl-coenzyme A dehydrogenase, very long chain (“Acadvl”), was found to be down regulated by compounds C1 and C2. Very-long-chain acyl-coenzyme A dehydrogenase (“VLCAD”) is a coenzyme encoded by Acadvl responsible for the conversion of very-long-chain fatty acids into energy. Genetic diseases linked to the deficiency of Acadvl are reported in many patients, as reported in the art.

On the other hand, acetyl-coenzyme A acetyltransferase 3 (“Acat2”) was found to be overexpressed by compound C1 more than compound C2. The gene is involved in intestinal cholesterol absorption and transport in chylomicrons and transferring CEs to VLDL in the liver, as reported in the art.

The results also show that acyl CoA thioesterase 12 (“Acot12”) was down-regulated by compounds C1 and C2. The gene encodes a major cytoplasmic enzyme in the liver. Acot12 contributes to the regulation of lipid biosynthesis by controlling the rate of degradation of cytosolic acetyl COA, especially in the liver, and inhibition of Acot12 transcription leads to a subsequent increase in cytosolic acetyl-CoA that protects against heart disease, as reported in the art.

Furthermore, Acyl-CoA synthetase long-chain family member 4 (“Acsl4”) gene expression was upregulated by compound C1 more than compound C2. Acsl4 encodes an enzyme with unique substrate specificity for arachidonic acid. Hepatic Acsl4 has an important role in in plasma TG, glucose metabolism and hepatic phospholipid synthesis of hyperlipidemia, as reported in the art.

Moreover, Acyl-CoA synthetase medium-chain family member 2 (“Acsm2a”) compound C1 was over expressed by compound C1 more than compound C2. The gene encodes an enzyme that catalyzes fatty acid activation, the first step of fatty acid metabolism, as reported in the art.

Additionally, 3-hydroxybutyrate dehydrogenase, type 2 (“Bdh2”) that encodes an enzyme mediating mitochondrial β-oxidation was found to be overexpressed by compound C1 more than compound C2. Bdh2 is a key regulator of de novo lipid biosynthesis, and mechanistically related to the development of hepatic insulin resistance and dyslipidemia, as reported in the art.

On the other hand, Carnitine palmitoyltransferase 1a, liver (“Cpt1a”) encodes the key enzyme inmitochondrial fatty acid oxidation was found to be down-regulated by both compounds C1 and C2.

Also, compound C1 down-regulated 2,4-dienoyl CoA reductase 1, mitochondrial (“Derc 1”), whereas compound C2 showed less than 2 folds down regulation. The protein encoded is involved in the degradation of long chain and very long chain fatty acyl-coenzyme A (“CoAs”) catalyzing the essential step for the β-oxidation of polyunsaturated fatty acids, and provides the requirement for a continual supply of fatty acids to support ongoing cell proliferation, also DecR1 deficiency results in the buildup of fatty acid intermediates due to incomplete β-oxidation, as reported in the art.

Fatty acid binding protein 7 (“Fabp7”) gene expression was down-regulated by both compounds C1 and C2. The protein encoded by this gene is one of several proteins important in long chain fatty acid uptake and metabolism in the hepatocytes, as reported in the art.

Fatty acid binding protein 4, adipocyte (“Fabp4”) was over expressed by compound C2, whereas compound C1 showed a change less than 2 in fold regulation. Over expression of Fabp4 was evaluated as a biomarker of metabolic and cardiovascular diseases in the art.

Both compounds C1 and C2 caused down regulation of Hydroxyacyl-Coenzyme A dehydrogenase/3-ketoacyl-Coenzyme A thiolase/enoyl-Coenzyme A hydratase (trifunctional protein), alpha subunit (“Hadha”) gene expression, which is reported in the art as a responsible for encoding a protein important in mitochondrial β oxidation.

Furthermore, 3-hydroxy-3-methylglutaryl-Coenzyme A synthase 1 (soluble) (“Hmgcs1”) was found to be over expressed by compound C2 more than compound C1, Hmgcs1 catalyzes the condensation of acetyl-CoA with acetoacetyl-CoA towards 3-hydroxy-3-methylglutaryl (HMG)-CoA.HMGCS1, the rate-limiting enzymes of the cholesterol biosynthesis pathway, as reported in the art.

Lipase, hormone sensitive (“Lipe”) was found to be over expressed by compounds C1 and C2. Lipe plays a pivotal role in providing the major source of energy for most tissuelt is reported in the art that Lipe is capable of converting cholesteryl esters to free cholesterol for steroid hormone production.

Compounds C1 and C2 caused Lipoprotein lipase (“LPL”) over expression., which encodes the primary enzyme responsible for the conversion of lipoprotein triglyceride into free fatty acids and monoglyderides. This permits their uptake into muscle and adipose. In addition, any genetic changes of LPL that result in human diseases, as reported in the art.

Methylmalonyl CoA epimerase (“Mcee”) encodes another enzyme that is important in mitochondrial β oxidation, and was down-regulated by compounds C1 and C2.

Solute carrier family 27 (fatty acid transporter), member 2 (“Slc27a2”) gene expression was down-regulated by both compounds C1 and C2. This protein plays a role in physiological and pathological situations which is implicated in lipid biosynthesis and fatty acid degradation. It is an early marker of obesity development related to the intake of a hyperlipidemic diet, as reported in the art.

Finally, (solute carrier family 27) (fatty acid transporter), member 5 (bile acid CoA ligase) (“Slc27a5”) was found to be down-regulated by compound C2 while compound C1 did not lead to 2-fold change in gene expression. The art reported that the protein encoded by this gene plays a crucial role in the liver and is involved in hepatic lipid and bile metabolism.

### Lipoprotein Signaling and Cholesterol Metabolism

In order to identify the molecular mechanism of the targeted compounds C1, C2, rat lipoprotein signaling and cholesterol metabolism profiler (PARN-0080ZA) (QUAIGEN, U.S.A.) was used in duplicates.

For each well of 96 wells from each PCR array, about 25 μl of PCR mix was added. After that, CFX96 real time PCR machine (Bio-Rad, U.S.A.) was used according to the following cycling conditions: about10 minutes at about 95° C., then about 15 seconds at about 95° C., and about 1 minute at about 60° C. for about 40 cycles, with adjusting to the RPM rate at about 1° C./s.

84 genes were analyzed for rat lipoprotein signaling and cholesterol metabolism PCR array. Tables (7) and (8) show genes overexpressed and down-regulated in Triton WR-1339 control group. Compounds C1, C2 show overexpressed and down regulation genes in comparison to Triton WR-1339 control group as shown in Table (9) below.

TABLE 7
Genes overexpressed in Triton WR-1339 control group
in lipoprotein signaling and cholesterol metabolism
Genes Overexpressed in Triton Control Group

| Gene symbol | Fold regulation |
|---|---|
| Apoa2 | 3.26 |
| Apof | 21 |
| Ebp | 14.33 |
| Apoa1 | 25.13 |
| Apoe | 744.94 |

TABLE 8
Gene down-regulated in Triton WR-1339 control group
in lipoprotein signaling and cholesterol metabolism
Genes down-regulated in Triton Control Group

| Gene symbol | Fold regulation |
|---|---|
| Cyp7a1 | −2.7 |
| Prkag2 | −2.21 |

TABLE 9
Gene expression in triton, compounds C1, and C2 in treated
rats in lipoprotein signaling and cholesterol metabolism

| Gene Name | Triton 1339 WR | Compound C1 | Compound C2 |
|---|---|---|---|
| Apoa2 | 3.26 | −1.29 | −5.47 |
| Apof | 21 | −1.47 | −2.14 |
| Cyp7a1 | −2.7 | 10.77 | 2.92 |
| Ebp | 14.33 | −1.42 | −2.76 |
| Osbpl5 | −1.97 | 4.43 | 4.09 |
| Prkag2 | −2.21 | 2.07 | 1.91 |
| Akr1d1 | 2.02 | −1.69 | −1.28 |
| Apoa1 | 25.13 | −1.69 | −5.3 |
| Apoe | 744.94 | 1.43 | −1.58 |

The results show that compound C2 down regulated Apolipoprotein A-II (“Apoa2”), while compound C1 did not affect the fold regulation of this gene by 2 folds or more. The protein produced is known in the art to have the main apolipoprotein found in HDL-C and is responsible for protective effect against atherosclerosis and used to be as a marker to assess in cardiovascular disease and therapeutic treatment.

Triton 1339 WR increased Apolipoprotein F (“Apof”) gene expression by 21 folds, this effect was reversed by compounds C1 and C2, ApoF is a protein component of several lipoprotein classes including HDL-C. Overexpression of ApoF reduces HDL cholesterol levels by increasing clearance of HDL-C, as reported in the art.

Results also show that Cyp7a1 was over expressed by compound C1 more than compound C2. The gene encodes the rate limiting enzyme and the major site in the regulation of bile acid synthesis, that converts cholesterol to bile acids, which is the primary mechanism for the removal of cholesterol from the body, as reported in the art.

Similar to Apof, emopamil-binding protein (“Ebp”) was upregulated by Triton 1339WR, but compounds C1 and C2 downregulated its expression. It is reported in the art that EBP is a high-affinity binding protein and belongs to the family called sigma receptors. The protein encoded plays a role in maintaining cholesterol homeostasis. thus, altered cholesterol homeostasis result in common disorders such as atherosclerotic cardiovascular disease and stroke.

Treatment with compounds C1 and C2 led to over expression of Osbp15. It is reported in the art that the gene encodes an enzyme that indicates several biochemical functions, for example, cholesterol binding, oxysterol binding, phosphatidylinositol-4-phosphate.

Furthermore, Protein kinase, AMP-activated, gamma 2 non-catalytic subunit (“Prkag2”) gene expression was up regulated by compound C1, but compound C2 did not produce a 2-fold change in its gene expression. It is reported in the art that Prkag2 plays a key role in regulating cellular energy metabolism, biosynthesis of fatty acid and cholesterol.

Moreover, Aldo-keto reductase family 1, member D1 (delta 4-3-ketosteroid-5-beta-reductase) (“Akr1d1”) was found to be down-regulated by both compounds C1 and C2, the gene is involved in bile acid biosynthesis, as reported in the art.

Apolipoprotein A1 (“ApoA1”) was found to be down-regulated by compounds C1 and C2. ApoA1 is reported in the art to have a protected effect against atherosclerosis and cardiovascular disease.

Apolipoprotein E (“Apoe”) was found to be over expressed by Triton 1339 WR and treatment with compounds C1 and C2 did not significantly affect the gene expression. It is reported in the art that the gene encodes an enzyme is an anti-atherogenic protein that plays a critical role in maintaining plasma cholesterol and triglyceride homeostasis.

The results show that there are over expressed and down regulated genes important in fatty acid metabolism and lipoproteins signaling metabolic pathways. Obviously, the process of hyperlipidemia development involves a variety overlapping pathways. Triton WR1339 has been found to over express several lipoproteins; every one of them has an atherogenic or antiatherogenic and the overall result shows the development of hypercholesterolemia and hypertriglyceridemia at the biological level.

At the molecular level, it appears that differences between Compound 1 and Compound 2 are not significant, so both show the same molecular behavior, commonly through over expression of fatty acid metabolizing enzymes and down regulating fatty acid and cholesterol biosynthesis.

While embodiments of the present disclosure have been described in detail and with reference to specific embodiments thereof, it will be apparent to one skilled in the art that various additions, omissions, and modifications can be made without departing from the spirit and scope thereof.

In describing and claiming the present invention, the following terminology will be used.

The singular forms “a,” “an,” and “the” include plural referents unless the context clearly dictates otherwise.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a defacto equivalent of any other member of the same list solely based on their presentation in a common group without indications to the contrary.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

What is claimed is:

1. A picolinic-2-carboxime compound of formula (I), or salts thereof: 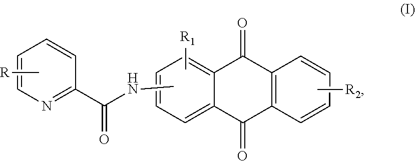
wherein
R is H, C1-C12 alkane, OH, O-(C1-C6) , NH₂, NHR⁴, R⁴ being C1-C6, or
R₁ is H, C1-C12 alkane, OH, O-(C1-C6), NH₂, NHR⁴, or halogen, and
R₂ is H, halogen, C1-12 alkane, OH, O-(C1-C6), NH₂, NHR⁴, CN, COOH, COOR⁴, or CONHR⁴.

2. A method of preparing the compound of claim 1, the method comprising:
mixing picolinic acid and thionyl chloride in toluene to produce a first mixture;
refluxing the first mixture till completion of reaction, thereby obtaining a refluxed first mixture;
adding toluene to the refluxed first mixture to produce a second mixture, then evaporating the second mixture under vacuum to remove thionyl chloride and produce picolinoyl chloride;
adding the picolinoyl chloride to an anthraquinone derviative, pyridine, and trimethylamine to produce a third mixture;
heating the third mixture to produce a solid mixture;
cooling down the solid mixture and adding cold water to the solid mixture while stirring, followed by adjusting the pH using carbonate to remove excess picolinoyl chloride, and to provide a fourth mixture; and
performing suction filtration to the fourth mixture in order to provide a fifth mixture comprising the picolinic-2-carboxime compound.

3. The method of claim 2, further comprising:
recrystallizing the fifth mixture from methanol.

4. The method of claim 2, wherein the anthraquinone derivative comprises 1-amino-anthraquinone.

5. The method of claim 2, wherein the anthraquinone derivative comprises 2-amino-anthraquinone.

6. The method of claim 2, wherein the anthraquinone derivative comprises 1-amino-4-hydroxy-amino-anthraquinone.

7. A pharmaceutical composition, comprising:
the picolinic-2-carboxime compound of claim 1; and/or
a pharmaceutically acceptable salt of the picolinic-2-carboxime compound; and
a pharmaceutically acceptable carrier/excipient.

8. The composition of claim 7, formulated as a solid, liquid, or semi-solid dosage form.

9. The composition of claim 7, which is configured for oral, parenteral, intramuscular, intranasal, sublingual, intratracheal, ocular, vaginal, rectal, or intraventricular administration.

10. A method for treating hyperlipidemia, the method comprising:
administering to a subject in need thereof an effective amount of the pharmaceutical composition of claim 7, thereby treating hyperlipidemia in the subject.

11. The compound of claim 1, wherein R₂ is the halogen, which is F or Cl.

12. The compound of claim 1, wherein R₂ is the halogen, which is I or Br.

13. The method of claim 2, wherein the refluxing of the first mixture is for 24 hours at a temperature of 80° C., and/or
wherein the heating of the third mixture is at a temperature of 100° C.

14. The method of claim 2, wherein the adjusting of the pH is to 10.

15. The method of claim 2, wherein the adjusting of the pH comprises adding potassium carbonate.

16. The compound of claim 1, wherein
R is H,
R₁ is OH, and
R₂ is H.

17. The compound of claim 1, wherein the picolinamide is bonded to the 2′-position on the anthraquinone.

18. The compound of claim 1, wherein the picolinamide is bonded to the 1′-position on the anthraquinone.

19. The compound of claim 1, wherein the picolinamide is bonded to the 1′-position on the anthroquinone, and
wherein R is H, R₁ is OH, and R₂ is H.

20. A picolinic-2-carboxime compound of formula (I), or salts thereof: 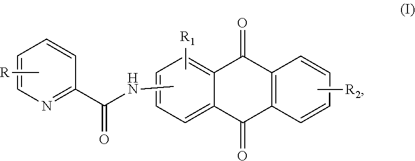
wherein
R is H, C1-C12 alkane, OH, O-(C1-C6), NH₂, NHR⁴, NO₂, R⁴ being methyl, ethyl, propyl, isopropyl, butyl, isobutyl, sec-butyl, or tert-butyl, or
R₁ is H, C1-C12 alkane, OH, O-(C1-C6), NH₂, NHR⁴, or halogen, and
R₂ is H, halogen, C1-12 alkane, OH, O-(C1-C6), NH₂, NHR⁴, CN, COOH, NO₂, COOR⁴, or CONHR⁴.
